(19) United States
(12) Patent Application Publication
(10) Pub. No.: US 2025/0344832 A1
(43) Pub. Date: Nov. 13, 2025

(54) ADHESIVE NUMBING PATCHES HAVING SHAPES WHICH CORRESPOND TO PARTICULAR AESTHETIC PROCEDURES

(71) Applicant: Root Cause Wellness and Aesthetics Inc., Napa, CA (US)

(72) Inventors: Nicholas Hounslow, Los Angeles, CA (US); Kristin Gisler, Vacaville, CA (US); Paula Rood, Napa, CA (US)

(73) Assignee: Root Cause Wellness and Aesthetics Inc., Napa, CA (US)

(21) Appl. No.: 18/658,926

(22) Filed: May 8, 2024

(51) Int. Cl. A45D 44/00; A61K 8/02

(52) U.S. Cl. CPC A45D 44/002; A61K 8/0212; A45D 2200/1036

(57) ABSTRACT

A numbing substance, such as lidocaine or other substances, is applied to the back surface of a silicone or fabric patch. The patch has a shape that depends on the particular cosmetic procedure to be performed. For a neck procedure, an arc-shaped numbing patch is provided to be applied around the neck. For an under-eye procedure, where the area to be treated is directly below the eye, the patch has a paisley or quotation mark shape. For a facial cosmetic procedure, the patch is a mask that contours to the face. For a lip procedure, the patches have a lip shape. The size of the patch is selectable, the numbing substance strength is selectable (e.g., from 5-25% potency), and the shape of the patch corresponds to the cosmetic procedure to be performed. The patches are supplied in air-tight foil packages.

### FIELD OF THE INVENTION

This invention relates to the application of a numbing agent to a person's skin and, in particular, to an adhesive applicator (a patch) having a shape that corresponds to an aesthetic (cosmetic) procedure to be performed.

### BACKGROUND

Lidocaine patches are commonly used to relieve pain in localized areas of the body. There are various other types of numbing substances that may also be provided by an adhesive patch. The numbing substance is designed provide a constant numbness over a period of time while the patch is secured to the skin. The patches are sold in generic geometric shapes, such as rectangular or circular. A backing is pealed off the patch, and an adhesive in the mixture causes the patch to firmly stick to the skin. Examples of use are for back pain, muscle pain, etc.

Such patches are not suitable for use before aesthetic procedures such as laser treatments or injections of substances under the skin. For such procedures, the numbing substance should be of a higher quality to start numbing within only a few minutes, and the area requiring numbing needs to be exposed to perform the aesthetic procedure. Conventional methods require a practitioner performing the aesthetic procedure to apply a non-predetermined amount of the numbing substance to an applicator, such as a tongue depressor, and then smear the substance on the patient's skin. Right before the aesthetic procedure, the practitioner wipes the substance off using gauze.

One issue with the conventional procedure is that the amount of numbing substance applied is not predetermined, resulting in varying levels of numbness for different patients. Another issue is that the application and removal of the numbing substance is messy. Another issue is that the numbing substance is wasted if too much is used. Another issue is that the numbing substance is typically applied to an area that is larger than needed, resulting in unnecessary numbness. Another issue is that the numbing substance can migrate into the patient's mouth, causing tongue, mouth, and throat numbness.

Accordingly, for suitable medical procedures, such as cosmetic procedures, what is needed is a new technique for applying a numbing substance to a patient's skin that: 1) applies an optimized predetermined amount of the numbing substance; 2) applies an optimized predetermined strength of the numbing substance, and; 3) applies the numbing substance to only the area that requires numbness for the particular cosmetic procedure.

### SUMMARY

A numbing substance, such as lidocaine or other substances, is applied to the back surface of a silicone or fabric patch. The patch has a shape that depends on the specific body area affected by the procedure. The examples given are for cosmetic procedures performed on the patient's neck, under-eyes, lips, nose, forehead, or larger portions of the face.

For a neck procedure, where the skin is tightened, only the area that will be injected or treated needs to be numbed, so an arc-shaped numbing patch is provided to be applied around the neck. For an under-eye treatment, where the area to be injected or treated is directly below the eye, the patch has a paisley or quotation mark shape. For an overall face cosmetic procedure, the patch is a mask that contours to the face. For a lip procedure, the patches correspond to the shape of lips.

The size of the patch is selectable from small to extra-large, the numbing substance strength is selectable (e.g., from 5-25% potency), and the shape of the patch corresponds to the procedure to be done. The patches are supplied in air-tight foil packages. The adhesive is inherent in the gel-like mixture, such as including silicone, so the adhesion is much weaker than in conventional lidocaine patches, since the patch is only for a short term in a medical office.

After numbing, the biodegradable patch is thrown away without any mess.

### BRIEF DESCRIPTION OF THE DRAWINGS

FIG. 1 illustrates a paisley-shaped patch applied to a patient's face that is used for under-eye cosmetic treatments.

FIG. 2 illustrates left and right patches that have been removed from an airtight pouch for under-eye cosmetic treatments.

FIG. 3 illustrates one of the patches of FIG. 2 being removed from a backing layer.

FIG. 4 illustrates an arc-shape of a patch that is used for neck cosmetic treatments.

FIG. 5 illustrates a person holding the patch of FIG. 4.

FIG. 6 illustrates the patch of FIG. 5 applied to a patient's neck.

FIG. 7 is a front view of a mask-shape of a patch that is used for facial cosmetic treatments.

FIG. 8 is a perspective view of the patch of FIG. 7.

FIG. 9 illustrates the patch of FIG. 8 applied over the patient's face.

FIG. 10 illustrates a lip-shaped patch.

FIG. 11 illustrates the patch of FIG. 10 applied over the patient's lips.

FIG. 12 illustrates the layers of any of the patches described herein.

Elements that are the same or equivalent are labelled with the same numerals.

### DETAILED DESCRIPTION

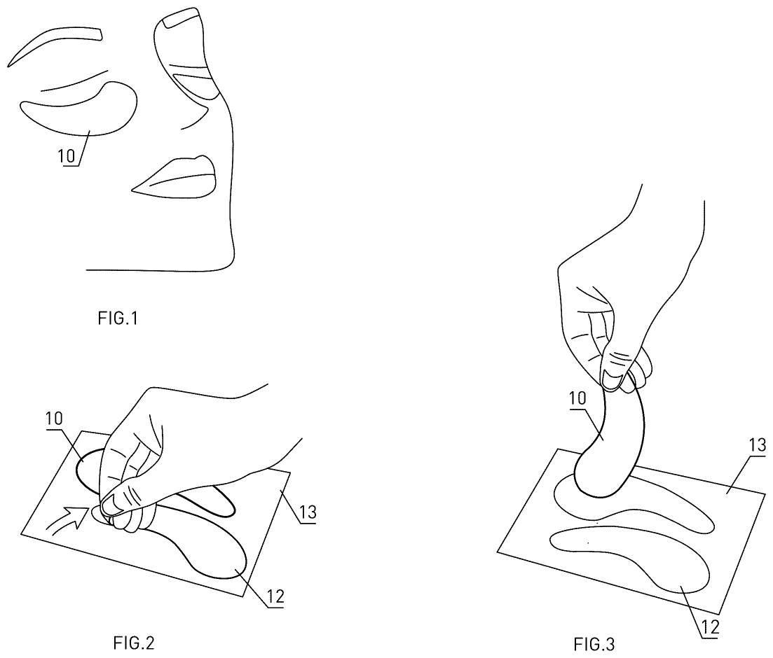

FIG. 1 illustrates one embodiment of the invention where a numbing patch 10 is weakly affixed below a patient's eye for an eye treatment, such as where the skin under the eye is injected or resurfaced with a laser. FIG. 2 shows a paisley-shaped left patch 10 and a right patch 12 on a backing layer 13 after being removed from an airtight package. The shape also resembles a question mark. FIG. 3 shows the patch 10 removed from the backing layer 13. The patches 10 and 12 come in different sizes (small, medium, large, and extra large) and are shaped to only cover the area to be numbed for a particular cosmetic treatment.

In one example, a thin layer of gel-like silicone is impregnated with a mixture of benzocaine, lidocaine, tetracaine, phenylephrine, and a liposome carrier that accelerates the numbing substance absorption into the skin. A heating agent may also be in the mixture for heating the mixture once exposed to air to speed up the numbing effect.

The impregnated silicone gel is applied in a predetermined thickness and strength (potency) to the surface of a flexible support layer, such as a fabric, paper, plastic, or silicone layer. A removable layer is provided over the impregnated silicone gel to prevent the gel from drying out and prevent it from contacting the inside of an airtight foil package for the patches.

The silicone gel weakly adheres to the patient's skin, and the characteristics of the numbing mixture are chosen to quickly numb the covered area with an optimal amount of the numbing substance. After a predetermined time, the patch 10 is removed and any residual numbing substance on the skin is wiped off.

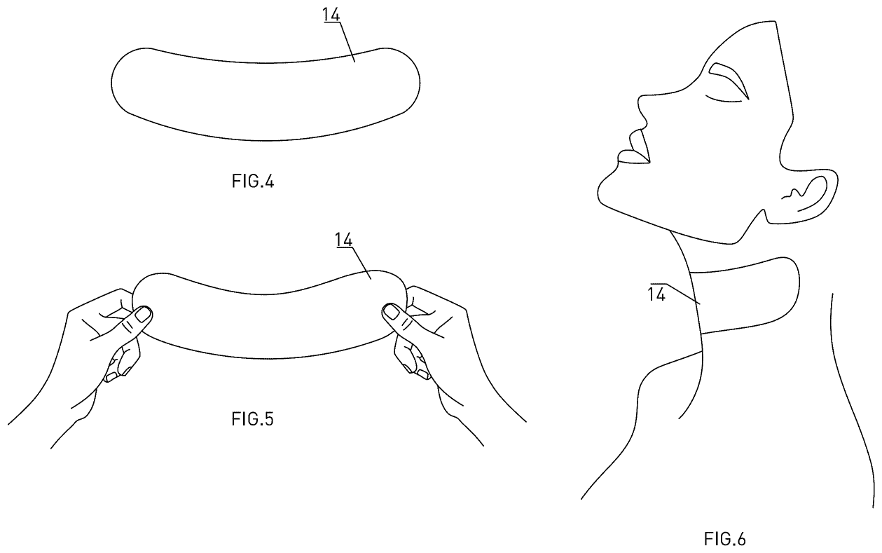

FIG. 4 illustrates a patch 14 that is formed in an arc shape for adhering around the patient's neck for a neck treatment. The curved shape results in the patch 14 covering a horizontal band of a predetermined width. The patches 14 are provided in multiple widths and lengths depending on the size of the neck and the procedure. FIG. 5 illustrates the practitioner holding the patch 14, and FIG. 6 illustrates the patch 14 adhered to the patient's neck.

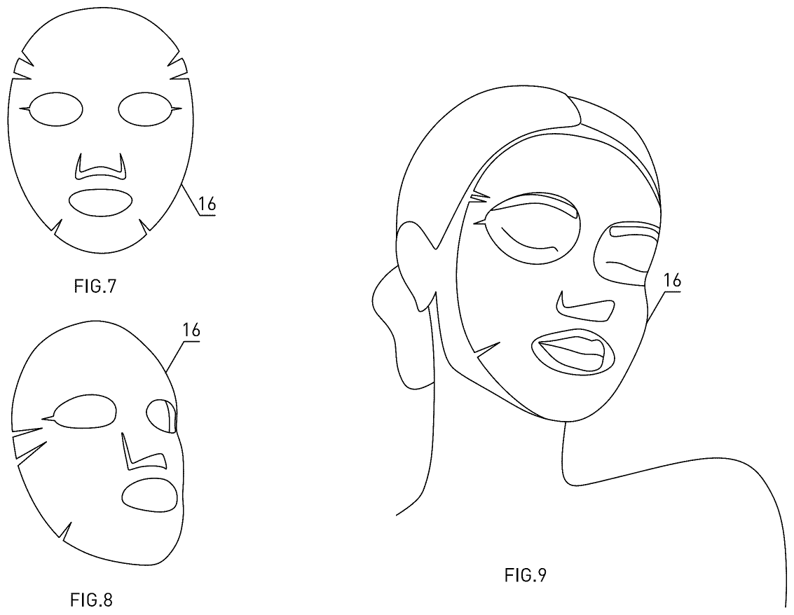

FIGS. 7, 8, and 9 illustrate a resilient mask-shaped patch 16 that covers the patient's face for an aesthetic procedure. The patch 16 may be provided flat in an airtight pouch and then bent over the face.

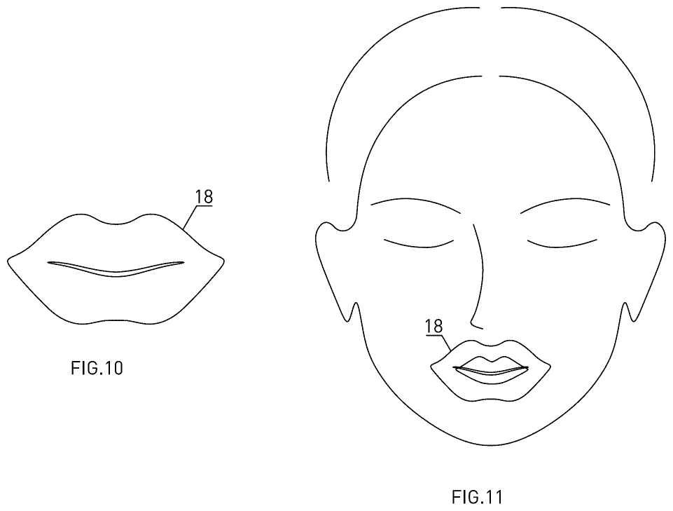

FIGS. 10 and 11 illustrate a lip-shaped patch 18 that covers the patient's lips for an aesthetic procedure, such as an injection.

Many other shapes are envisioned for different cosmetic procedures. Other shapes include a nose-shaped patch and a forehead-shaped patch.

Some examples of cosmetic treatments that may be performed on different parts of the patient's face, neck, and lips include injecting neurotoxins, injecting fillers, injecting PRP/PRF, injecting peptides, injecting exosomes, laser treatments, microneedling, PDO thread lifts, and nitrogen plasma treatments.

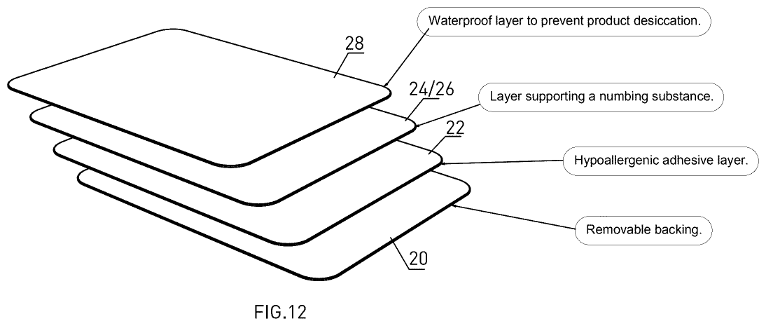

FIG. 12 illustrates the layers of any one of the patches, where a flexible backing layer 20, such as a fabric, paper, plastic, or silicone layer, has a hypoallergenic adhesive layer 22 applied over it. A flexible support sheet 24, such as a fabric, paper, plastic, or silicone layer, has applied to its bottom surface a numbing substance 26 having a predetermined thickness and strength (potency). The numbing substance may be a combination of known materials used for numbing. An optional waterproof layer 28 is applied over the support sheet 24 to prevent the numbing substance 26 from drying out. The strength, sizes, and shapes are selectable, and each patch or set of patches is provided in its own airtight foil package until needed. The layers may be cut in a desired shape (FIGS. 1-11) so as to be removable from a rectangular backing layer 20.

The numbing substance 26 may include a heating agent for faster absorption by the skin. Such a numbing substance may be Synera™ by Johnson & Johnson, which includes lidocaine, tetracaine, and an oxygen-activated heating agent. A Synera patch is described in U.S. Pat. No. 6,465,709, incorporated herein by reference.

This concept of shaped patches customized for a particular procedure can also be applied to non-cosmetic surgeries.

While particular embodiments of the present invention have been shown and described, it will be obvious to those skilled in the art that changes and modifications may be made without departing from this invention in its broader aspects and, therefore, the appended claims are to encompass within their scope all such changes and modifications as fall within the true spirit and scope of this invention.

What is claimed is:

1. A numbing patch comprising:
a flexible support layer having a shape that corresponds with a particular type of treatment to be performed, the shape being other than rectangular or circular; and
a numbing substance deposited on the support layer.

2. The patch of claim 1 wherein the shape comprises a paisley shape for under-eye treatment.

3. The patch of claim 1 wherein the shape comprises an arc shape for neck treatment.

4. The patch of claim 1 wherein the shape comprises a face-mask shape for facial treatment.

5. The patch of claim 1 wherein the shape comprises a lip shape for lip treatment.

6. The patch of claim 1 wherein the numbing substance comprises silicone, which provides a weak adhesion to a person's skin.

7. The patch of claim 1 where the patch is selected from an assortment of patches having different sizes, shapes, and numbing substance strengths.

8. The patch of claim 1 where the patch is configured for use with cosmetic facial treatments.

9. The patch of claim 8 where the patch is configured for use with treatments comprising at least one of injecting neurotoxins, injecting fillers, injecting PRP/PRF, injecting peptides, injecting exosomes, laser treatments, microneedling, PDO thread lifts, and nitrogen plasma treatments.

10. A method of performing a facial cosmetic procedure comprising:
selecting a numbing patch from an assortment of patches having different sizes and shapes, the numbing patch comprising a flexible support layer having a shape that corresponds with a particular type of cosmetic procedure to be performed, the shape being other than rectangular or circular, and a numbing substance deposited on the support layer;
applying the patch to an area of a patient's body to be numbed;
removing the patch after a period of time; and
performing the cosmetic procedure after the patch has been removed.

11. The method of claim 10 wherein the assortment of patches also has different numbing substance strengths.

12. The method of claim 10 wherein the shape comprises a paisley shape for under-eye treatment.

13. The method of claim 10 wherein the shape comprises an arc shape for neck treatment.

14. The method of claim 10 wherein the shape comprises a face-mask shape for facial treatment.

15. The method of claim 10 wherein the shape comprises a lip shape for lip treatment.

16. The method of claim 10 wherein the numbing substance comprises silicone, which provides a weak adhesion to a person's skin.

17. The method of claim 10 where the patch is configured for use with treatments comprising at least one of injecting neurotoxins, injecting fillers, injecting PRP/PRF, injecting peptides, injecting exosomes, laser treatments, microneedling, PDO thread lifts, and nitrogen plasma treatments.
